(19) United States
(12) Patent Application Publication
(10) Pub. No.: US 2010/0094344 A1
(43) Pub. Date: Apr. 15, 2010

(54) Pedicle-Based Posterior Stabilization Members and Methods of Use

(75) Inventor: Hai H. Trieu, Cordova, TN (US)

Correspondence Address: MEDTRONIC;Attn: Noreen Johnson - IP Legal Department, 2600 Sofamor Danek Drive, MEMPHIS, TN, 38132, US

(73) Assignee: KYPHON SARL, Neuchatel (CH)

(21) Appl. No.: 12/250,678

(22) Filed: Oct. 14, 2008

(51) Int. Cl. A61B 17/70; A61B 17/04

(52) U.S. Cl. 606/246; 606/278; 606/301; 606/305

(57) ABSTRACT

The present application is directed to pedicle-based posterior stabilization members and methods of stabilizing vertebral members. The stabilization members generally include a body with first and second connector sections. The first connector section is configured to connect to a pedicle of a first vertebral member. The second connector section is configured to contact an adjacent vertebral member. The second connector section may be positioned at an end of the body, or an intermediate section away from the end.

### BACKGROUND

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Spinal implants are often used in the surgical treatment of spinal disorders such as degenerative disc disease, disc herniations, curvature abnormalities, and trauma. Many different types of treatments are used. In some cases, spinal fusion is indicated to inhibit relative motion between vertebral members. In other cases, dynamic implants are used to preserve motion between vertebral bodies. In yet other cases, relatively static implants that exhibit some degree of flexibility may be attached to the vertebral members.

The spinal implants may provide a stable, rigid column that encourages bones to fuse after spinal-fusion surgery. Further, the implants may redirect stresses over a wider area away from a damaged or defective region. Also, an implant may restore the spine to its proper alignment.

Aside from each of these characteristic features, a surgeon may wish to control anatomic motion after surgery. That is, a surgeon may wish to inhibit or limit one type of spinal motion while allowing a lesser or greater degree of motion in a second direction. As an illustrative example, a surgeon may wish to inhibit or limit motion of lateral bending while allowing a greater degree of flexion and extension.

### SUMMARY

The present application is directed to stabilization members to stabilize first and second vertebral members. The stabilization member may include an elongated rod with a first end and a second end. The rod may include an arcuate shape to position the first end at a pedicle of the first vertebral member and a second connector section near the second end at either the lamina or the spinous process of the second vertebral member. The rod may be attached to the first vertebral member at the first connector section and unattached from the first connector section to the second end with the second end being free to move relative to the second vertebral member. The rod may be shaped with the second end in closer proximity to a midline of the first and second vertebral members than the first end.

The various aspects of the various embodiments may be used alone or in any combination, as is desired.

### BRIEF DESCRIPTION OF THE DRAWINGS

FIG. 1 is a schematic rear view of a stabilization member engaging first and second vertebral members according to one embodiment.

FIG. 2 is a perspective view of a fastener attaching a stabilization member to a vertebral member according to one embodiment.

FIG. 3 is a side view of a first connector section of a stabilization member according to one embodiment.

FIG. 4 is a side view of a second connector section of a stabilization member according to one embodiment.

FIG. 5 is a schematic rear view of a stabilization member engaging first and second vertebral members according to one embodiment.

FIG. 6 is a schematic side view of a stabilization member engaging first and second vertebral members according to one embodiment.

FIG. 7 is a schematic rear view of a stabilization member engaging first and second vertebral members according to one embodiment.

FIG. 8 is a schematic rear view of a stabilization member engaging first and second vertebral members according to one embodiment.

FIG. 9 is a schematic rear view of first and second stabilization members engaging first and second vertebral members according to one embodiment.

FIG. 10 is a schematic rear view of first and second stabilization members engaging first and second vertebral members according to one embodiment.

FIG. 11 is a schematic rear view of first and second stabilization members engaging first and second vertebral members with a bumper positioned between the stabilization members according to one embodiment.

FIG. 12 is a side view of a stabilization member according to one embodiment.

FIG. 13 is a side view of a stabilization member according to one embodiment.

FIG. 14 is a schematic rear view of a stabilization member engaging first and second vertebral members according to one embodiment.

FIG. 15 is a schematic rear view of a stabilization member engaging first and second vertebral members according to one embodiment.

### DETAILED DESCRIPTION

The present application is directed to pedicle-based posterior stabilization members and methods of stabilizing vertebral members. The stabilization members generally include a body with first and second connector sections and an intermediate section. The first connector section is configured to connect to a pedicle of a first vertebral member. The second connector section is configured to contact an adjacent vertebral member. The second connector section may be positioned at an end of the body, or an intermediate section away from the end. The body includes a second end that is free such that it is not connected with a fastener to either the first or second vertebral members.

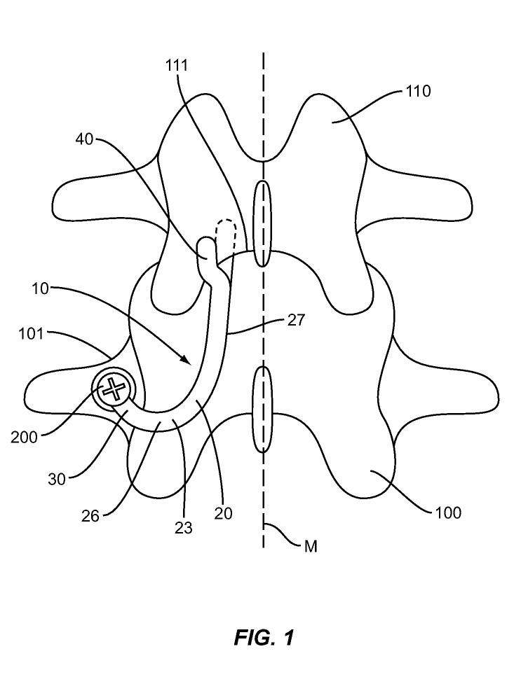

FIG. 1 illustrates a stabilization member 10 with the body 20 with a first connector section 30 connected to a first vertebral member 100 and a second connector section 40 contacting a second vertebral member 110. An intermediate section 23 is positioned between the connector sections 30, 40. The body 20 may be constructed from various materials including but not limited to metal, polymers, ceramics, and combinations thereof. Examples of metals include titanium, titanium alloys such as nickel-titanium, stainless steel, and cobalt chromium. Examples of polymers include PEEK, PEEK-carbon composites, polyimide, polyetherimide, and polyurethane. Examples of ceramics include calcium phosphate, hydroxyapatite, HAPCP, alumina, and zirconium.

The body 20 may include different characteristics depending upon the desired result. The body 20 may be rigid, flexible, elastic, and inelastic. These characteristics may be achieved by one or more of the material itself, the cross-sectional shape of the body 20, and the cross-sectional size of the body 20. The body 20 may include a substantially constant cross-sectional shape throughout the length. The body 20 may also include one or more sections with a reduced cross-sectional size that causes flexibility and elasticity at these sections. The body 20 may also include one or more sections with different cross-sectional shapes that again provide flexibility and elasticity to specific sections.

The body 20 may be constructed to provide dynamic stabilization while maintaining certain biomechanical motions of the spine. Stabilization may be maintained during motions such as extension, lateral bending, and rotation. The body 20 may also be constructed to have a predetermined stiffness to provide the stabilization. Further, the stiffness may vary during a biomechanical motion. The body 20 may further be constructed to not interfere with flexion.

FIG. 1 includes the stabilization member 10 operatively connected to first and second vertebral members 100, 110. The body 20 is configured to place the first connector section 30 at the pedicle 101 of the first vertebral member 100 and the second connector section 40 at the lamina 111 of the second vertebral member 110. The first connector section 30 is fixedly connected to the pedicle 101 such that the body 20 supports the second vertebral member 110 through the second connector section 40. The second end 22 of the body 21 is free such that it is not fixedly connected to the second vertebral member 110.

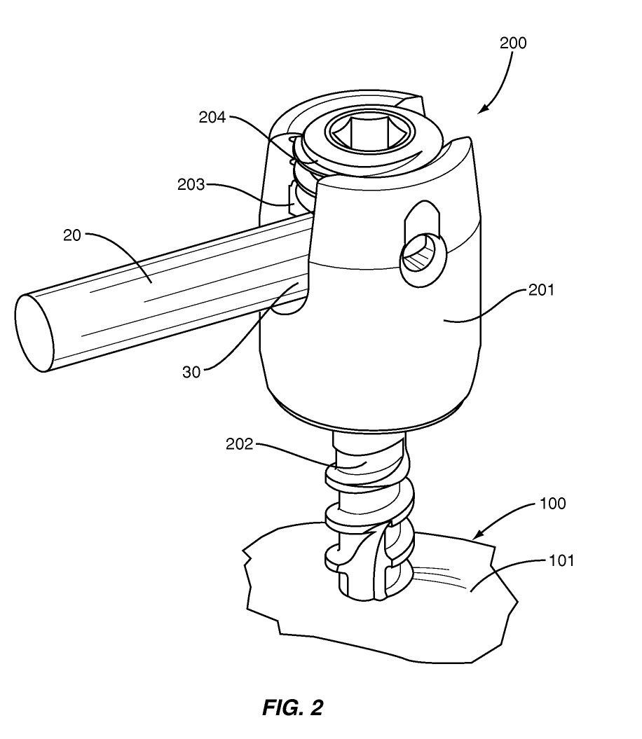

In this embodiment, the first connector section 30 is fixed within a pedicle screw 200 connected to the pedicle 101 of the first vertebral member 100. FIG. 2 illustrates a fastener 200 that connects the first connector section 30 of the body 20 to the pedicle 101 of the first vertebral member 100. The fastener 200 includes a receiver 201 with a channel 203 sized to receive the body 20. A set screw 204 attaches to the receiver 201 to capture the body 20 within the channel 203. Fastener 200 also includes an anchor 202 that attaches to the pedicle 101 of the first vertebral member 100. The fastener 200 may be constructed for the receiver 201 to move relative to the anchor 202 (i.e., multi-axial fastener) to allow for multi-axial positioning of the body 20. The receiver 201 may also be fixed relative to the anchor 202 (i.e., mono-axial). This type of fastener 200 illustrated in FIG. 2 provides for adjusting an effective length because the first connector section 30 may be moved within the channel 203 as necessary to position the second connector section 40. One embodiment of a fastener is disclosed in U.S. patent Ser. No. 12/038,572 filed Feb. 27, 2007 and herein incorporated by reference.

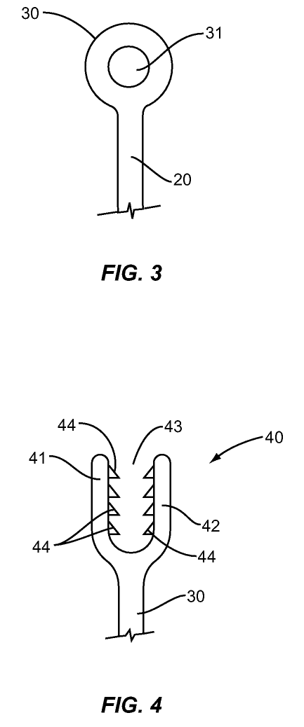

FIG. 3 illustrates another embodiment with the first connector section 30 comprising a ring 31 formed at the end of the body 20. The ring 31 is sized to receive a fastener (not illustrated) to connect to the pedicle 101 of the first vertebral member 100.

The first connector section 30 is often positioned at an end of the body 20. FIGS. 2 and 3 each illustrate embodiments with this configuration. Other embodiments may include the first connector section 30 positioned within an intermediate section of the body 20 between the ends. One example may include an embodiment similar to FIG. 2 except that the body 20 extends through the channel 203 and the end of the body 20 is positioned outward from the receiver 201.

The second connector section 40 is in contact with and supports the second vertebral member 110. As with the first connector section 30, the second connector section 40 may be positioned at the end of the body 20, or at an intermediate section of the body 20 spaced from the end. The second connector section 40 is not fixedly attached to the second vertebral member 110.

FIGS. 1 and 4 each illustrate a second connector section 40 comprising a jaw formed by first and second arms 41, 42 that are spaced apart to form a channel 43. The channel 43 is sized to receive a section of the second vertebral member 110. In the embodiment of FIG. 1, the channel 43 is sized to receive the lamina 111 of the second vertebral member 110. The first and second arms 41, 42 may include the same or different lengths. The distance between the arms 41, 42 forming the width of the channel 43 may also vary depending upon the context of use. Teeth 44 may be positioned along the inner edges of one or both arms 41, 42. The teeth 44 facilitate engagement between the second connector section 40 and the second vertebral member 110. The teeth 44 may further include angled surfaces that allow movement of the second vertebral member 110 into the channel 110, and resist movement out of the channel 110. The channel 43 may also include a cushioning layer to soften the contact with the second vertebral member 110. Cushioning material such as plastics and elastomers may be attached to the inner surfaces of the arms 41, 42 to provide the cushioning.

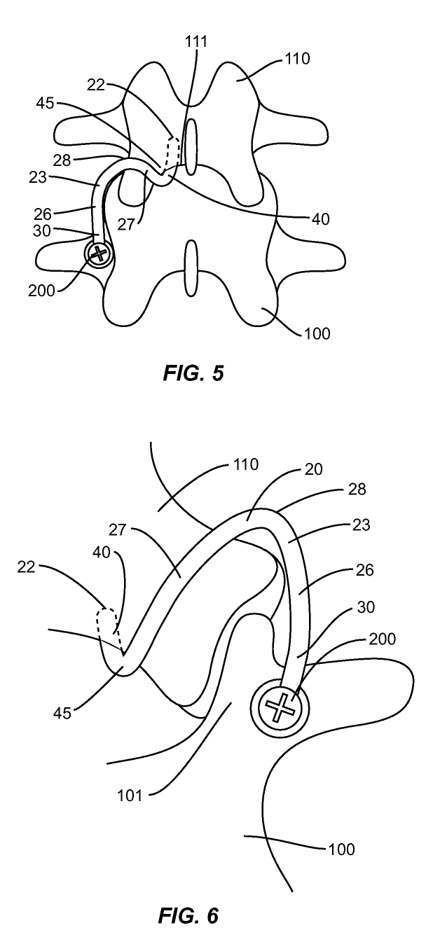

FIG. 5 includes the second connector section 40 formed by a bend 45 in the body 20. The bend 45 is sized and configured such that a first section of the body 20 is positioned on a posterior side of the second vertebral member 110 and a second section is positioned on an anterior side. The bend 45 is positioned away from the second end 46 of the body 20. The bend 45 is positioned such that the second section of the body 20 on the anterior side is long enough to ensure the bend 45 remains positioned on the vertebral member 110 and does not slip off.

FIG. 6 includes a lateral view of a stabilization member 10 with the first connector section 30 attached with a fastener 200 to the pedicle 101 of a first vertebral member 100. The second connector section 40 is configured with a bend 45 that wraps around the lamina 111 of the second vertebral member 110. The intermediate section 23 of the body 20 between the first and second ends 21, 22 is configured to position these sections 20, 30 at the desired locations.

The intermediate section 23 of the body 20 is positioned between the first and second ends 21, 22. The intermediate section 23 is configured to position the first and second connector sections 30, 40 at the appropriate locations and also accommodate the physical structure of the first and second vertebral members 100, 110 and the surrounding tissue. The intermediate section 23 may include different configurations depending upon the specific context of use.

FIG. 1 includes the intermediate section 23 with a first lateral section 26 that extends from the first connector section 30 to a midpoint of the body 20. The intermediate section 23 also includes a second vertical section 27 that extends to the second connector section 40. FIG. 5 includes a configuration with the first section 26 extending along the sagittal plane with the second section 27 extending along the coronal plane. In this embodiment, the intermediate section 23 includes a bend 28 that is beyond the bend 45 of the second connector section 40 in the sagittal plane. FIG. 6 includes the body 20 configured with a bend 28 along the intermediate section 25 being beyond the bend 45 of the second connector section 40 in the sagittal plane.

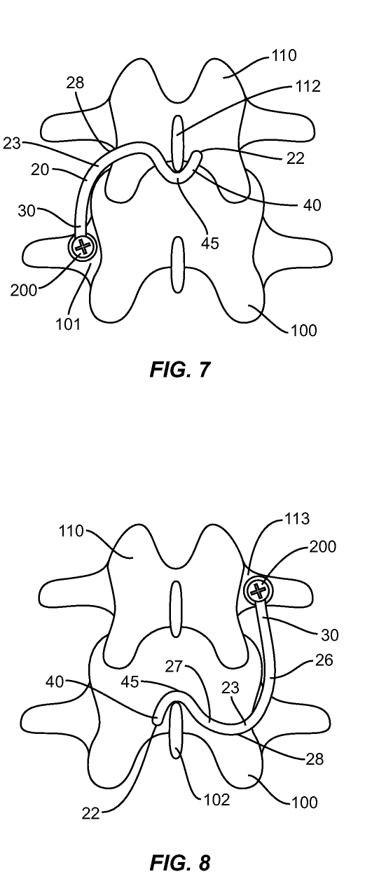

The stabilization member 10 may be configured for the second connector section 40 to contact the lamina of the second vertebral member 110. Examples of this are illustrated in FIGS. 1, 5, and 6. FIG. 7 includes the second connector section 40 contacting the spinous process 112 of the second vertebral member 110. The body 20 is configured with the second connector section 40 including a bend 45 positioned and configured to engage the spinous process 112. Specifically, the bend 45 contacts against an inferior surface of the spinous process 112. The first connector section 30 is attached with a fastener 101 to the pedicle 101 of the first vertebral member 100. The body 20 is further configured with the bend 28 of the intermediate section 25 above the bend 45 in the sagittal plane of the second connector section 40.

FIG. 8 includes an embodiment with the first section 30 connected with a fastener 200 to the pedicle 113 of the second superior vertebral member 110. The second section 40 includes a bend 45 that contacts the spinous process 102 of the first inferior vertebral member 100. Specifically, the bend 45 contacts against the superior surface of the spinous process 102. In this embodiment, the first section 30 is attached to the superior second vertebral member 110 and the second section 40 is attached to the inferior first vertebral member 100.

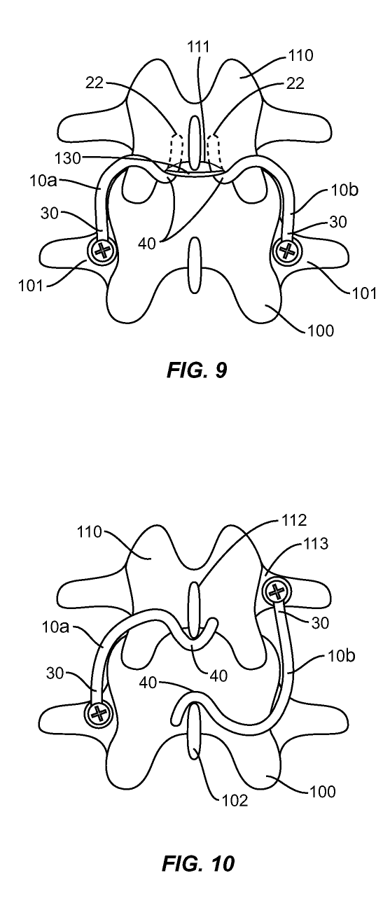

More than one stabilization member 10 may be used to stabilize the vertebral members 100, 100. FIG. 9 includes a first stabilization member 10a positioned on a first lateral side of the vertebral members 100, 110, and a second stabilization member 10b positioned on a second lateral side. Each member 10a, 10b includes a first connector section 30 attached to pedicles 101 of the first vertebral member 100, and a second connector section 40 that contacts against the lamina 111 of the second vertebral member 110. FIG. 10 includes another embodiment with a first stabilization member 10a on a first lateral side of the vertebral members 100, 110. The first stabilization member 10a includes a first connector section attached to the pedicle 101 of the first vertebral member 100 and a second connector section 40 contacting against an inferior surface of the spinous process 112 of the second vertebral member 110. The second stabilization member 10b includes a first connector section 30 attached to the pedicle 113 of the second vertebral member 110 and a second connection section 40 in contact with a superior surface of the spinous process 102 of the first vertebral member 100.

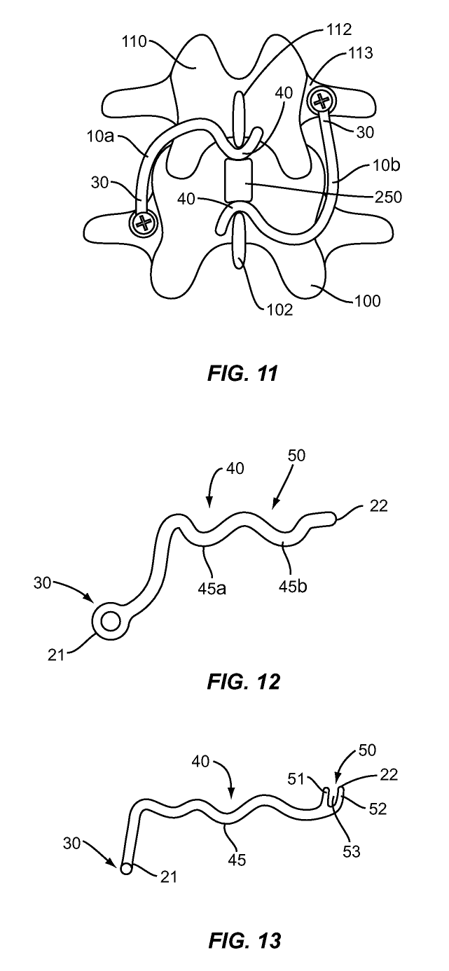

FIG. 11 illustrates a bumper 250 positioned between the first and second stabilization members 10a, 10b. The bumper 250 includes a stiffness to provide resistance during movement of the stabilization members 10a, 10b. The bumper 250 may be made from a deformable material that may stretch during vertebral flexion and/or compress during vertebral extension. The bumper 250 may also be relatively rigid to prevent flexion or extension, or limit the amount of movement beyond a predetermined amount. The bumper 250 may be attached to each stabilization member 10a, 10b in various manners, including but not limited to tethers, adhesives, mechanical fasteners. Ends of the bumper 250 may correspond in shape to the stabilization members 10a, 10b to facilitate attachment and/or positioning.

Bumper 250 may be constructed of a variety of different materials. Bumper 250 may be resilient and change shape during movement of the stabilization members 10a, 10b. Examples of such materials include elastic or rubbery polymers, hydrogels or other hydrophilic polymers, or composites thereof. Particularly suitable elastomers include silicone, polyurethane, copolymers of silicone and polyurethane, polyolefins, such as polyisobutylene and polyisoprene, neoprene, nitrile, vulcanized rubber and combinations thereof. Examples of polyurethanes include thermoplastic polyurethanes, aliphatic polyurethanes, segmented polyurethanes, hydrophilic polyurethanes, polyether-urethane, polycarbonate-urethane and silicone polyetherurethane. Other suitable hydrophilic polymers include polyvinyl alcohol hydrogel, polyacrylamide hydrogel, polyacrylic hydrogel, poly(N-vinyl-2-pyrrolidone hydrogel, polyhydroxyethyl methacrylate hydrogel, and naturally occurring materials such as collagen and polysaccharides, such as hyaluronic acid and cross-linked carboxyl-containing polysaccharides, and combinations thereof.

A cross-link member 130 may connect together two or more of the stabilization members 10a, 10b as illustrated in FIG. 9. The cross-link member 130 extends between and provides support to each of the stabilization members 10a, 10b. The cross-link member 130 may be positioned at various locations along the lengths of the stabilization members 10a, 10b from the first end 21 to the second end 22. Further, multiple cross-link members 130 may extend between the stabilization members 10a, 10b.

The stabilization members 10 may also include multiple connector sections that are spaced away from the first connector section 30. FIG. 12 includes an embodiment with connector sections 40, 50 each configured to contact against one of the vertebral members 100, 110. Connector section 40 is formed by a first bend 45a and the connector section 50 is formed by a second bend 45b. In one embodiment, the bends 45a, 45b are spaced apart to engage with different sections of the lamina 111. FIG. 13 includes an embodiment with connector section 40 formed by a bend 45 and another connector section 50 formed as a jaw by spaced apart arms 51, 52 that form a channel 53. Additional embodiments may include stabilization members 10 with more that two connector sections spaced away from the first connector section 30.

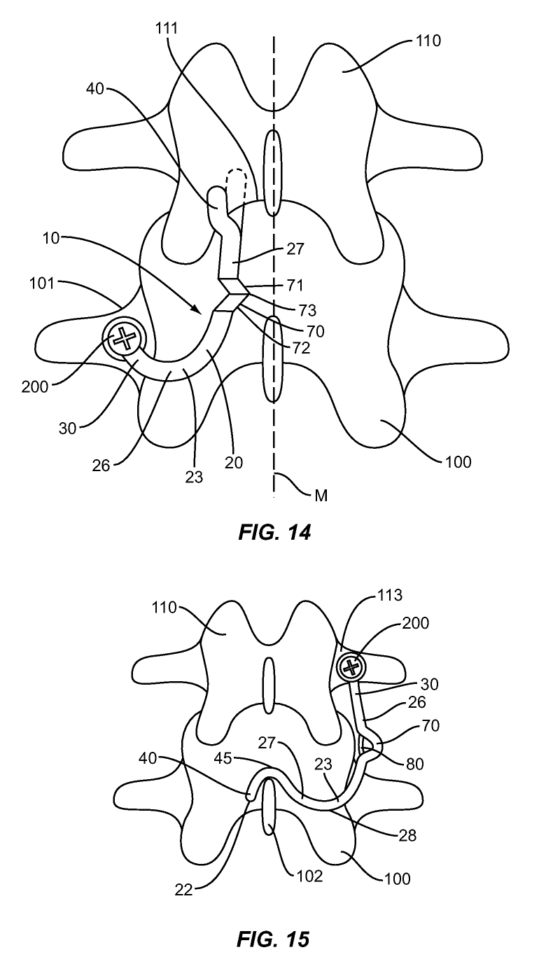

A flexible section 70 may be positioned along the body 20 between the first connector section 30 and the second connector section 40 as illustrated in FIG. 14. The flexible section 70 includes opposing sections that are movable during vertebral movement to provide variable resistance. The variable resistance may provide dynamic stabilization during a normal range of motion for the neutral position during flexion, extension, lateral bending, and rotation.

The flexible section 70 may include various structures that form the opposing sections. FIG. 14 includes opposing first and second sections 71, 72 that are connected along a fold 73. During vertebral movement, the sections 71, 72 may move towards and away from each other providing the variable resistance and dynamic stabilization. The sections 71, 72 may be formed from the same or different material as the body 20. The flexible section 70 may also include one section with a more continuous curve when viewed from a side such as a C-shape or V-shape. The flexible section 70 may also include a serpentine shape with multiple curves and multiple vertical overlapping sections.

An elastic member 80 may be positioned in the flexible section 70 as illustrated in FIG. 15. The member 80 may include a stiffness to provide resistance to movement of the opposing portions of the flexible section 70. The elastic member 80 may impose a substantially linear or non-linear resistance to movement of the flexible section 70. The elastic member 80 may be sized to connect to the flexible section 70 at two opposing positions. Member 80 may also connect to a single position along the flexible section 70. The elastic member 80 may be sized to fill a portion or the entirety of the area between the opposing sections 71, 72. The elastic member 80 may be loaded in compression and/or tension. The member 80 may be attached to the flexible section 70 in various manners, including adhesives, and mechanical fasteners such as screws, pins, rivets, and the like.

Elastic member 80 may be constructed of a variety of different materials. Member 80 may be resilient and change shape during movement of the sections 20, 30. Examples of such materials include elastic or rubbery polymers, hydrogels or other hydrophilic polymers, or composites thereof. Particularly suitable elastomers include silicone, polyurethane, copolymers of silicone and polyurethane, polyolefins, such as polyisobutylene and polyisoprene, neoprene, nitrile, vulcanized rubber and combinations thereof. Examples of polyurethanes include thermoplastic polyurethanes, aliphatic polyurethanes, segmented polyurethanes, hydrophilic polyurethanes, polyether-urethane, polycarbonate-urethane and silicone polyetherurethane. Other suitable hydrophilic polymers include polyvinyl alcohol hydrogel, polyacrylamide hydrogel, polyacrylic hydrogel, poly(N-vinyl-2-pyrrolidone hydrogel, polyhydroxyethyl methacrylate hydrogel, and naturally occurring materials such as collagen and polysaccharides, such as hyaluronic acid and cross-linked carboxyl-containing polysaccharides, and combinations thereof.

Embodiments of the flexible section and elastic member are disclosed in U.S. Patent Publication 2007/0191832 herein incorporated by reference.

The second connector sections 40 may include osteoconductive and/or osteoinductive materials to facilitate the contact with the vertebral member 100, or 110.

FIG. 1 includes a posterior view of the first and second vertebral members 100, 110. A midline M extends through the vertebral members 100, 110. The stabilization member 10 is configured when attached to the vertebral members 100, 110 with the first section 26 being substantially perpendicular to the midline M. The second section 27 is substantially parallel to a midline M.

The stabilization member 10 may be used on adjacent vertebral levels. Embodiments are illustrated in FIGS. 1 and 5-10 with the first connector section 30 attached to vertebral member 100 and the second connector section 40 attached to adjacent vertebral member 110. The stabilization member 10 may also be configured to skip one or more vertebral levels. By way of example, the first connector section 30 may attach to the L4 vertebral member and the second connector section 40 may attach to the L2 or L1 vertebral members. The stabilization member 10 may be used along the various sections of the spine.

The stabilization member 10 may be implanted within a living patient for the treatment of spinal disorders such as degenerative disc disease, disc herniations, curvature abnormalities, and trauma. The stabilization member 10 may provide greater amount of spinal movement for the patient and/or reduce or eliminate pain. The stabilization member 10 may also be implanted in a non-living situation, such as within a cadaver, model, and the like. The non-living situation may include attachment of the first connector section 30 to a first vertebral member 100 and the second connector section 40 to a second vertebral member 110. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A stabilization member to stabilize first and second vertebral members comprising:
an elongated rod with a first end and a second end and a bent intermediate section positioned between the first and second ends;
a first connector section positioned in proximity to the first end, the first connector section including a connection configured to be attached to a pedicle of the first vertebral member; and
a second connector section positioned in closer proximity to the second end than to the first end, the second connector section configured to engage an inferior surface of one of a lamina and spinous process of the second vertebral member;
a section of the rod from the first connector section to the second end being unattached to either of the first and second vertebral members.

2. The stabilization member of claim 1, wherein the intermediate section includes a reduced cross-sectional area that is smaller than at least one of the first end and the second end.

3. The stabilization member of claim 1, wherein the intermediate section includes a cross-sectional shape that is different than at least one of the first end and the second end.

4. The stabilization member of claim 1, further comprising a flexible section positioned along the rod between the first and second connector sections, the flexible section including opposing sections that are configured to move relative to each other during movement of the first and second vertebral members and an elastic member attached to the flexible section and positioned between the opposing sections.

5. The stabilization member of claim 1, wherein the second connector section is positioned at the second end of the rod.

6. The stabilization member of claim 5, wherein the second connector section comprises first and second arms that are spaced apart to form a channel therebetween that is sized to receive the second vertebral member.

7. The stabilization member of claim 1, wherein the second connector section includes a bend formed along the rod at a point inward from the second end.

8. The stabilization member of claim 1, wherein a section of the rod between the bent intermediate section and the second end is substantially parallel to a midline of the first and second vertebral members.

9. The stabilization member of claim 1, wherein a section of the rod between the bent intermediate section and the first end is substantially perpendicular to a midline of the first and second vertebral members.

10. A stabilization member to stabilize first and second vertebral members comprising:
an elongated rod with a first end, a second end, a first connector section, and a second connector section, the rod including an arcuate shape to position the first connector section and the first end at a pedicle of the first vertebral member and the second connector section at one of a lamina and spinous process of the second vertebral member;
a connector to attach the first connector section to the pedicle of the first vertebral member;
a majority of a length of the rod from the connector to the second end being free such that the second connector section contacts the second vertebral member and may move relative to the second vertebral member.

11. The stabilization member of claim 10, wherein the second connector section is positioned at the second end of the rod.

12. The stabilization member of claim 10, further comprising a flexible section positioned between the first and second connector sections, the flexible section including opposing sections with an elastic member positioned therebetween.

13. The stabilization member of claim 10, wherein the second connector section includes a bend in the rod with a posterior section configured to be positioned on a posterior side of the second vertebral member and an anterior section configured to be positioned on an anterior side of the second vertebral member.

14. A stabilization member to stabilize first and second vertebral members comprising:
an elongated rod with an arcuate shape to position a first end at a pedicle of the first vertebral member and a second end at an inferior surface of one of the second vertebral member lamina and spinous process;
a second connector section positioned in closer proximity to the second end than to the first end, the second connector section configured to engage one of a lamina and spinous process of the second vertebral member;
the first end configured to be attached to the pedicle of the first vertebral member and the second end being free to move relative to the second vertebral member;
the rod shaped with the second end in closer proximity to a midline of the first and second vertebral members than the first end.

15. The stabilization member of claim 14, wherein the second connector section comprises a jaw with first and second arms that are spaced apart to form a channel therebetween.

16. A method of stabilizing first and second vertebral members comprising:
attaching a first end of an elongated rod to a pedicle of the first vertebral member at an attachment location;
positioning an intermediate section of the rod along a posterior of one or both of the first and second vertebral members;
contacting a connector section of the rod to the second vertebral member, the connector section being spaced apart from the first attachment location; and
maintaining free a length of the elongated rod between the attachment location and the second end including the connector section.

17. The method of claim 16, further comprising positioning a section of the rod along an anterior side of the second vertebral member.

18. The method of claim 16, wherein contacting the connector section of the rod to the second vertebral member comprising contacting a lamina of the second vertebral member with a jaw at a second end of the rod.

19. The method of claim 16, further comprising contacting a second connector section of the rod to the second vertebral member, the second connector section being offset along the rod from the first connector section.

20. The method of claim 16, further comprising attaching the first end of the rod with a multi-axial connector to the pedicle of the first vertebral member.
